(19) United States
(12) Patent Application Publication
(10) Pub. No.: US 2025/0359612 A1
(43) Pub. Date: Nov. 27, 2025

(54) COMPRESSION GARMENT

(71) Applicant: Coria Skin Technologies Limited, London (GB)

(72) Inventors: Andrew WILEBORE, Leicestershire (GB); Stephen BARKER, Surrey (GB)

(21) Appl. No.: 18/873,719

(22) PCT Filed: Jun. 8, 2023
(86) PCT No.: PCT/GB2023/051492

§ 371 Date: Dec. 10, 2024

(30) Foreign Application Priority Data
Jun. 10, 2022 (GB) 2208541.9

(51) Int. Cl. A41D 31/18; A41D 1/04; A41D 1/089

(52) U.S. Cl. CPC A41D 31/185; A41D 1/04; A41D 1/089; A41D 2400/32; A41D 2400/38

(57) ABSTRACT

A compression garment configured to be worn over at least a wearer's pelvic area and upper legs is disclosed comprising an upper thigh region, a lower thigh region and a buttock region. An inner surface of each of the upper thigh, lower thigh, and buttock regions comprises a respective plurality of pads. Also disclosed is a compression garment configured to be worn over at least a wearer's shoulders and upper arms, comprising, an upper inner biceps region, an upper inner triceps region, and an outer arm region. An inner surface of each of the upper inner biceps, upper inner triceps and outer arm regions comprises a respective plurality of pads.

### BACKGROUND

Cellulite refers to the dimpling of skin (often termed “orange peel” skin) most often seen in areas with more significant fat deposition underlying such as the thighs, lower torso and upper arms. It is extremely prevalent in women (sometimes considered to affect more than 80% of women, and often affecting women in the younger age ranges from 12-45) but can also affect men.

Commonly cited causes of cellulite include disturbance of hormone levels (e.g. oestrogens), genetic propensity, obesity, rapid weight gain, patho-physiological links to 1o fluid/lymph retention, over-full fat cells, micro-vascular dysfunction and fibrosis (as fibrous bands of tissue in the sub-dermal fat layer).

While cellulite is generally categorised as a harmless skin condition, it can have significant detrimental mental health effects, including loss of self-esteem and anxiety over appearance.

Many potential (and often invasive) treatments have been proposed to reduce cellulite, although very few preventative treatments have been proposed. Skin specialists generally accept that once cellulite is present, it is virtually impossible to remove. While a myriad of oils, creams and gels are available, these generally have limited demonstrable effect backed by scientific studies and publications. Physical treatments are also available, including infra-red heat, acoustic wave therapy, local wraps providing a localised sauna-type heating (and sweating) effect, and surgical cutting of fibrous bands beneath the skin surface. However, there is again limited demonstrable and lasting effect backed by scientific studies and publications.

There has been an increase in recent years in the availability and variety of apparels, or specialist garments, that allegedly manage cellulite. Anti-cellulite apparels include leggings, jeggings, cropped leggings, cycle shorts, high-waisted briefs/pants and abdominal wraps. Many of these specialist garments utilise low level compression and/or link body re-shaping wear to cellulite management. Once again, there is limited demonstrable and lasting effect backed by scientific studies and publications. Most recently, some specialist garments are using textured compression fabrics.

Rather than attempting to treat/remove existing cellulite, there is a need for apparels/garments that help prevent cellulite forming and manage existing cellulite to avoid it becoming more severe.

### SUMMARY OF THE INVENTION

According to a first aspect of the invention, there is provided a compression garment comprising more than one compression region, an inner surface of each compression region comprising a respective plurality of pads, wherein the inter-pad spacings between each pad in the respective plurality of pads of neighbouring compression regions differs.

A compression garment is a garment which applies a compression load (force) on to the wearer's skin, and the compression garment may be a low-compression garment (i.e. having a sub-medical compression level of for example between 3 mmHg and 15 mmHg). The inner surface is the surface of the garment that faces the wearer's skin when the garment is being worn. The upper leg is defined herein as the part of the leg above the knee.

The present invention is based on consideration of two key concepts surrounding the formation of cellulite: (1) that micro-vascular (capillary) dysfunction leads to a poorer supply of Oxy-Haemoglobin (Oxy-Hb) to the skin and subcutaneous tissues (promoting with time fibrin deposition, fibrosis and fluid retention), and (2) that poor lymph (comprising protein-rich fluid and cellular ‘waste products’) drainage can contribute too, to the development of generalised tissue fibrosis and in particular, to those fibrous bands that form between subcutaneous fat (lipid-rich) cells (lipocytes). As these bands pass between them, to anchor on to the dermal (skin) layer above and to muscle sheaths and fascial layers below, and as those fat cells increase in volume and bulge/round up (hypertrophy) for any other reason, because the fibrous bands linked to the overlying dermal (skin) layer stay the same length, so the overlying ‘orange peel’ skin dimpling pattern becomes manifest.

Garments according to the present invention improve micro-vascular dysfunction and lymph drainage and may thereby help to both improve the appearance of already established cellulite and slow or prevent cellulite formation and progression.

The pads of the present invention have the effect of directionally ‘micro-massaging’ the skin and underlying dermal layer where cellulite forms and progresses. The pads enhance the passage of blood through the micro-vasculature, encouraging the delivery of well oxygenated and nutrient-rich blood. The terminology “pads” is used herein to refer to protruding elements on the inner surface of the garment, but the pads could also be referred to using alternative language such as “bumps”, “protrusions”, or “raised dots”.

The different pad spacings direct and encourage (or enhance) the passage of lymph along its ‘usual’ pathways (from more distally, towards more proximally) and so, help prevent the effects of poorly draining lymph. For example, the pads on the buttock region encourage both the delivery of Oxy-Hb to the overlying skin and simultaneously help the flow of lymph away from the skin and underlying dermis and towards the perineum (crotch) and so on to the groin lymph nodes.

Alternatively, all of the pads may be spaced with the same inter-pad spacings, such that the compression garment relies primarily on just the micro-massaging effect.

In some examples, the inter-pad spacings are between 2.5 mm and 5.5 mm. This allows inter-pad spacings to vary between regions without becoming too closely or widely spaced.

In some examples, the heights of each pad in the respective plurality of pads of neighbouring compression regions differs. That is to say, the heights as well as the inter-pad spacings vary between regions. Differential pad heights provide the same effects as differential inter-pad spacings, as will be discussed herein.

In some examples, the heights of the pads is between 0.2 mm and 1.5 mm. These pad heights provide a balance between wearer comfort and ensuring that the garment functions effectively. If the pads are much higher comfort may be reduced. If the pads are lower, then the garment will not be as effective at mitigating cellulite.

In some examples, the garment is configured to be worn over at least a wearer's pelvic area and upper legs, wherein the neighbouring compression regions include an upper thigh region, a lower thigh region and a buttock region,

In some examples, the inter-pad spacings of the respective plurality of pads on the upper thigh region are closer than the inter-pad spacings of the respective pluralities of pads in the lower thigh region and the buttock region.

In some examples, the garment further comprises a mid-thigh region, wherein the mid-thigh region comprises a plurality of pads, and wherein the inter-pad spacings of the plurality of pads in the mid-thigh region are wider than the inter-pad spacings of the respective plurality of pads in the upper thigh region and closer than the inter-pad spacings of the respective pluralities of pads in the lower thigh region and the buttock region.

Preferably, heights of the respective plurality of pads on the upper thigh region are lower than heights of the respective pluralities of pads in the lower thigh region and the buttock region. The term “heights of the respectively plurality of pads” refers to the mean height of pads in the respective region.

The different height regions direct and encourage the passage of lymph, similarly to different inter-pad spacings in between regions.

Alternatively, all of the pads may be the same height, such that the compression garment relies primarily on just the micro-massaging effect.

In some examples, the heights of the plurality of pads in the mid-thigh region are higher than the heights of the respective plurality of pads in the upper thigh region and lower than the heights of the respective pluralities of pads in the lower thigh region and buttock region. The mid-thigh region compresses the wearer's mid-thigh and improves lymph drainage in this area of the wearer's body. The intermediate heights of the pads in the mid-thigh region further graduate the pressure differential between the upper and lower thigh regions. This encourages the passage of lymph from the lower to upper front thigh through the front mid-thigh. On the back of the thigh, heights of the plurality of pads in the mid-thigh region are lower than heights of pads in the buttock region and the lower thigh region, encouraging the passage of lymph from the buttock and back lower thigh to the mid-thigh, and from the back mid-thigh to the front upper thigh.

The mid-thigh region is a region of the garment that covers at least part of the wearer's thigh between the upper thigh region and lower thigh region and/or between the lower thigh region and the buttock region.

In some examples, the garment is configured to be worn over at least a wearer's shoulders and upper arms, wherein the neighbouring compression regions include an upper inner biceps region, an upper inner triceps region and an outer arm region.

In some examples, the inter-pad spacings of the respective pluralities of pads on the upper inner biceps region and upper inner triceps region are closer than the inter-pad spacings of the respective plurality of pads in the outer arm region. This directs and encourages (or enhances) the passage of lymph along its ‘usual’ pathways (from more distally, towards more proximally) and so, help prevent the effects of poorly draining lymph in the upper body.

In some examples, the garment comprises a mid-biceps region, wherein the mid-biceps region comprises a plurality of pads, and wherein inter-pad spacings of the plurality of pads in the mid-biceps region are wider than the inter-pad spacings of the respective plurality of pads in the upper inner biceps region and closer than the inter-pad spacings of the respective pluralities of pads in the outer arm region. The intermediate spacings of the pads in the mid-triceps region further graduate the pressure differential between the upper inner triceps and outer arm regions. This encourages the passage of lymph from the outer arm to the regional axillary (armpit) lymph nodes through the upper inner triceps.

In some examples, the garment comprises a mid-triceps region, wherein the mid-triceps region comprises a plurality of pads, and wherein the inter-pad spacings of the plurality of pads in the mid-triceps region are wider than the inter-pad spacings of the respective plurality of pads in the upper inner triceps region and closer than the inter-pad spacings of the respective pluralities of pads in the outer arm region.

In some examples, the heights of the respective pluralities of pads on the upper inner biceps region and upper inner triceps region are lower than the heights of the respective plurality of pads in the outer arm region.

In some examples, the heights of the plurality of pads in the mid-biceps region are higher than the heights of the respective plurality of pads in the upper inner biceps region and lower than the heights of the respective plurality of pads in the outer arm region.

In some examples, the heights of the plurality of pads in the mid-triceps region are higher than the heights of the respective plurality of pads in the upper inner triceps region and lower than the inter-pad spacings of the respective pluralities of pads in the outer arm region.

In some examples, the garment has a compression level between 1 mmHg and 50 mmHg (inclusive), and even more preferably between 3 mmHg and 15 mmHg (inclusive) (between 3 mmHg and 15 mmHg is understood to be “low-compression”, i.e. sub-medical). This compression level ensures that the pads make sufficient contact with the skin for the garment to function correctly whilst maintaining comfort for the wearer.

According to a second aspect, there comprises a compression garment comprising more than one compression region, an inner surface of each region comprising a respective plurality of pads, wherein the heights of each pad in the respective plurality of pads of neighbouring compression regions differs.

According to a further aspect, there comprises a compression garment configured to be worn over at least a wearer's pelvic area and upper legs, comprising: an upper thigh region; a lower thigh region; and a buttock region, wherein an inner surface of each of the upper thigh, lower thigh, and buttock regions comprises a respective plurality of pads, wherein heights of the respective plurality of pads on the upper thigh region are lower than heights of the respective pluralities of pads in the lower thigh region and the buttock region.

In some examples, the garment comprises a mid-thigh region, wherein the mid-thigh region comprises a plurality of pads, and wherein heights of the plurality of pads in the mid-thigh region are higher than the heights of the respective plurality of pads in the upper thigh region and lower than heights of the respective pluralities of pads in the lower thigh region and buttock region.

In some examples, the heights of the plurality of pads in the mid-thigh region are between 0.2 to 4.0 mm (inclusive), more preferably between 0.3 and 1.5 mm (inclusive), and most preferably between 0.5 and 1.0 mm (inclusive). The pad heights are defined in the condition where the garment is unstretched and not being worn. When worn, the heights of some pads may change as those pads are stretched or deformed.

In some examples, the heights of the of the plurality of pads in the upper thigh region are 0.1 and 2.0 mm (inclusive), more preferably between 0.2 and 1.0 mm (inclusive), and most preferably between 0.3 and 0.5 mm (inclusive).

In some examples, the heights of the pluralities of pads in the lower thigh region and buttock region are between 0.3 and 6.0 mm (inclusive), more preferably between 0.6 and 3.0 mm (inclusive), and most preferably between 0.9 and 1.5 mm (inclusive).

These pad heights provide a balance between wearer comfort and ensuring that the garment functions effectively. If the pads are higher, comfort may be reduced. If the pads are lower, then the garment will not be as effective at mitigating cellulite.

In some examples, the garment comprises an unpadded groin region and/or an unpadded inside thigh region and/or an unpadded crotch region and/or an unpadded natal cleft region.

Preferably, the compression garment further comprises an unpadded groin region. An unpadded region is a smooth region that does not comprise pads on the inner surface of the garment. Unpadded regions are preferably left in regions near any named, superficial veins (so avoiding any named superficial lymphatic vessel(s) running alongside), to encourage lymphatic drainage from the smallest lymphatic vessels and plexuses towards the larger ‘collecting’ lymphatics and on towards the regional lymph nodes. Additionally, unpadded regions enhance wearer comfort by leaving sensitive regions such as the groin and crotch uncovered by pads.

Preferably, the compression garment is any one of a pair of: shorts, cycle shorts, trousers, leggings, cropped leggings, jeggings, briefs, or pants. The garment may be nightwear, e.g. trousers in the form of pajama bottoms.

According to a further aspect, there comprises a compression garment configured to be worn over at least a wearer's shoulders and upper arms, comprising: an upper inner biceps region; an upper inner triceps region; and an outer arm region, wherein an inner surface of each of the upper inner biceps, upper inner triceps and outer arm regions comprises a respective plurality of pads, wherein heights of the respective pluralities of pads on the upper inner biceps region and upper inner triceps region are lower than heights of the respective plurality of pads in the outer arm region.

As with the first aspect described above, the second aspect of the invention improves micro-vascular function and lymph drainage and thereby helps both improve the appearance of already established cellulite and slow or prevent cellulite formation and progression. The pads of the present invention have the effect of ‘micro-massaging’ the skin and underlying dermal layer where cellulite forms and progresses. The pads therefore enhance the passage of blood through the micro-vasculature, encouraging the delivery of well oxygenated and nutrient-rich blood. The terminology “pads” is used herein to refer to protruding elements on the inner surface of the garment, but the pads could also be referred to using alternative language such as “bumps”, “protrusions”, or “raised dots”.

The upper arm is the part of the arm above the elbow.

The upper inner biceps region is a region of the garment that covers at least part of the upper inner part of a wearer's biceps on the front of the arm when the garment is worn.

The upper inner triceps region is a region of the garment that covers at least part of the upper inner part of a wearer's triceps on the back of the arm when the garment is worn.

The outer arm region is a region of the garment that covers at least part of the outside (i.e. the lateral side) of the wearer's upper arm when the garment is worn. It may also be referred to as an outer upper arm region.

In some examples, the garment further comprises a mid-biceps region, wherein the mid-biceps region comprises a plurality of pads, and wherein heights of the plurality of pads in the mid-biceps region are higher than the heights of the respective plurality of pads in the upper inner biceps region and lower than heights of the respective pluralities of pads in the outer arm region. The mid-biceps region is a region of the garment that covers at least part of the wearer's biceps between the upper inner biceps region and outer arm region. The mid-biceps region compresses the wearer's mid-biceps and improves lymph drainage in this area of the wearer's body. The intermediate heights of the pads in the mid-biceps region further graduate the pressure differential between the upper inner biceps and outer arm regions. This encourages the passage of lymph from the outer arm to the regional axillary (armpit) lymph nodes through the upper inner bicep. The term “heights of the respectively plurality of pads” refers to the mean height of pads in the respective region. Alternatively, all of the pads may be the same heights, such that the compression garment relies primarily on the micro-massaging effect.

The different height regions direct and encourage (or enhance) the passage of lymph along its ‘usual’ pathways (from more distal to more proximal) and so, help prevent the effects of poorly draining lymph. For example, the pads on the upper biceps encourage both the delivery of Oxy-Hb to the overlying skin and underlying dermis and simultaneously help the flow of lymph away from the skin and underlying dermis towards the shoulder and so on to the regional axillary (armpit) lymph nodes.

In some examples, the heights of the pluralities of pads in the mid-biceps region are between 0.2 and 4.0 mm (inclusive), preferably between 0.3 mm and 2.0 mm (inclusive), and most preferably between 0.5 and 1.0 mm (inclusive). The pad heights are defined in the condition where the garment is unstretched and not being worn. When worn, the heights of some pads may change as those pads are stretched or deformed.

In some examples, the heights of the pluralities of pads in the upper inner biceps region and upper inner triceps region are between 0.1 and 2.0 mm, preferably between 0.2 and 1.0 mm, and most preferably between 0.3 and 0.5 mm.

In some examples, the heights of the plurality of pads in the outer arm region are between 0.3 and 6.0 mm, preferably between 0.6 and 3.0 mm, and most preferably between 0.9 and 1.5 mm.

In some examples, the garment comprises an unpadded torso region, and/or an unpadded inside arm region. An unpadded region is a smooth region that does not comprise pads on the inner surface of the garment.

Unpadded regions are preferably left in regions near any named, superficial veins (so avoiding any named superficial lymphatic vessel(s) running alongside), to encourage lymphatic drainage from the smallest lymphatic vessels and plexuses towards the larger ‘collecting’ lymphatics and so on towards the regional (axillary) lymph nodes. Additionally, unpadded regions enhance wearer comfort by leaving sensitive regions such as the inside arm and armpit uncovered by pads.

In some examples, the garment is anyone selected from a list consisting of: a t-shirt, a polo shirt, a cycling vest, a jumper, a sweater, a fleece, a shirt, and a blouse.

In some examples wherein a spacing between edges of neighbouring pads in each region is between 1 and 5 mm and preferably between 1.5 and 3 mm.

In some examples, the garment has a compression level between 1 mmHg and 50 mmHg, and preferably between 3 mmHg and 15 mmHg.

According to a further aspect, there comprises a compression garment configured to be worn over at least a wearer's pelvic area and upper legs comprising a plurality of padded regions, wherein when the compression garment is worn, the plurality of padded regions is arranged to push lymph along a plurality of natural drainage paths from the wearer's lower thigh and buttock to a lymph node in the wearer's front upper 1o thigh.

According to a further aspect, there comprises a compression garment configured to be worn over at least a wearer's shoulders and upper arms comprising a plurality of padded regions, wherein when the compression garment is worn, the plurality of padded regions is arranged to push lymph along a plurality of natural drainage paths from a lower area of the wearer's upper arm to a lymph node in the wearer's armpit.

According to a further aspect, there comprises a method of manufacturing a compression garment comprising: providing a compression fabric; screen-printing respective pluralities of ink polymer pads in regions of the compression fabric to form neighbouring compression regions, wherein the number of layers forming the respective ink polymer pads in each region differs; and forming the compression fabric into a shape suitable to be worn as a garment.

According to a further aspect, there comprises a method of manufacturing a compression garment, comprising: providing a compression fabric; screen-printing a first plurality of ink polymer pads in an upper thigh region of the compression fabric, each of the first plurality of ink polymer pads having at least one layer; screen-printing a second plurality of ink polymer pads in a mid-thigh region of the compression fabric, each of the second plurality of ink polymer pads having at least two layers (and preferably at least one more layer than each of the first plurality of ink polymer pads); screen-printing a third plurality of ink polymer pads in a buttock region and a lower thigh region of the compression fabric, each of the second plurality of ink polymer pads having at least three layers (and preferably at least one more layer than each of the second plurality of ink polymer pads); and forming the compression fabric into a shape suitable to be worn over at least a wearer's pelvic area and upper legs. (e.g. by sewing, gluing, or ultrasonic welding). The compression fabric may be formed into the shape of the compression garment before or after the pads are applied to the fabric.

According to a further aspect, there comprises a method of manufacturing a compression garment, comprising: providing a compression fabric; screen-printing a first plurality of ink polymer pads in an upper inner biceps region and an upper inner triceps region of the compression fabric, each of the first plurality of ink polymer pads having at least one layer; screen-printing a second plurality of ink polymer pads in a mid-biceps region and a mid-triceps region of the compression fabric, each of the second plurality of ink polymer pads having at least two layers (and preferably at least one more layer than each of the first plurality of ink polymer pads); screen-printing a third plurality of ink polymer pads in an outer arm region of the compression fabric, each of the second plurality of ink polymer pads having at least three layers (and preferably at least one more layer than each of the second plurality of ink polymer pads); and forming the compression fabric into a shape suitable to be worn over at least a wearer's shoulders and upper arms comprising a plurality of padded regions. (e.g. by sewing, gluing, or ultrasonic welding). The compression fabric may be formed into the shape of the compression garment before or after the pads are applied to the fabric.

In some examples, the pads are (substantially) cylindrical in shape. This cylindrical shape allows the pads to be more easily manufactured (e.g. using a screen-printing process) and also avoids sharp/pointed edges/corners that would reduce wearer comfort. The pads may alternatively have an oval cross section (i.e. they may be an oval cylinder shape). Alternatively, the pads may have some other shape, such as dome shaped or U-shaped.

The pads may be distributed in an ordered pattern over each region, or they may be distributed stochastically (randomly).

### BRIEF DESCRIPTION OF THE DRAWINGS

Examples of the present invention will now be described in detail with reference to the accompanying drawings, in which:

FIGS. 1A-C show a compression garment configured to be worn over at least a wearer's pelvic area and upper legs, illustrating the location of regions on the inner side of the garment;

FIGS. 2A-B show an embodiment of the compression garment in which the garment has been turned inside-out to show the inner surface and the regions with respective pluralities of pads thereon;

FIGS. 3A-C show the compression garment of FIG. 1, with arrows illustrating the egress of lymph in the wearer's body;

FIGS. 4A-B show a plurality of regions of a simplified garment, each region comprising a plurality of pads, such that the average pad height varies between regions;

FIGS. 5A-B show an alternative embodiment of a compression garment;

FIGS. 6A-C show a compression garment configured to be worn over at least a wearer's shoulders and upper arms, illustrating the location of regions with differing pad heights on the inner side of the garment; and

FIGS. 7A-B show the compression garment of FIG. 6, with arrows illustrating the egress of lymph in the wearer's body.

FIG. 8A-B are graphs showing the results of a trial to evaluate a compression garment.

FIGS. 9A-B show a plurality of regions of a simplified garment, each region comprising a plurality of pads, such that the average inter-pad spacing varies between regions.

FIGS. 10A-B shows a template for screen-printing onto the inner surface of a leg of a compression garment. FIG. 10A shows the entire template, while FIG. 10B shows only the front portion of the template.

FIGS. 11A-C show a compression garment configured to be worn over at least a wearer's shoulders and upper arms, illustrating the location of regions with differing inter-pad spacings on the inner side of the garment.

### DETAILED DESCRIPTION

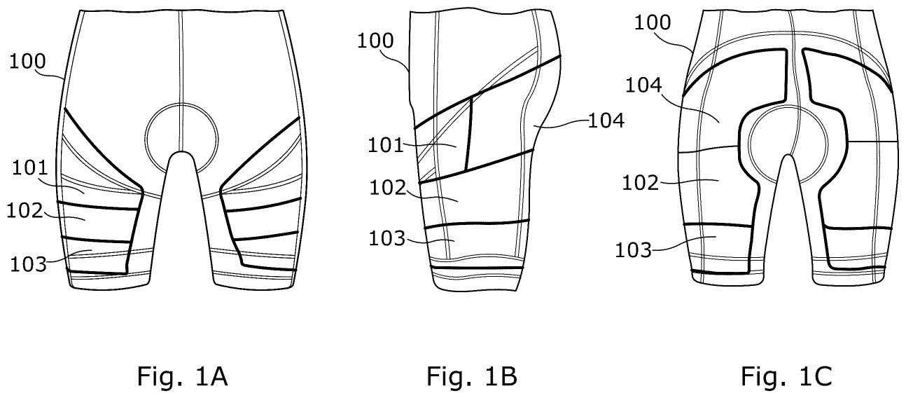

FIGS. 1A, B, and C show a front view, side view, and back view respectively of a compression garment 100 configured to be worn over at least a wearer's pelvic area and upper legs. The garment 100 in this embodiment is a pair of shorts, which cover the wearer's body from the waist down to the top of the knees.

Four padded regions are illustrated on the inner surface of the garment 100. An upper thigh region 101 covers the wearer's front upper thigh, a mid-thigh region 102 covers the front, outside and rear of the wearer's mid-thigh, a lower-thigh region 103 covers the front, outside and rear of the wearer's lower thigh, and a buttock region 104 covers the wearer's buttock. Alternative embodiments are also envisioned in which one or more of the regions 101-104 are larger or smaller and/or have different shapes (for example, the mid-thigh region 102 may only cover the front of the wearer's thigh, and the buttock region 104 may extend down the rear of the thigh to top of the lower thigh region 103). In addition, one or more of the regions (in particular the mid-thigh region 102) could be omitted in alternative embodiments, and the size and shape of the remaining regions could be adjusted accordingly (e.g. the lower thigh region 103 may be extended to the bottom of the buttock region 104 and upper thigh region 101).

The illustrated regions are symmetric on the wearer's left and right sides. Each padded region 101-104 comprises a plurality of pads (the pads themselves are not shown in FIGS. 1A, B and C) on the inner surface of the garment 100 (the inner surface being the surface of the garment 100 that faces the wearer's skin when the garment 100 is being worn). The regions 101-104 may be contiguous as shown in FIGS. 1A-C and 2A and B, or alternatively some of the regions 101-104 may be separated from each other by spaces which do not have pads. Heights of the pads in the upper thigh region 101 are lower than heights of the pads in the mid-thigh region 102. Heights of the pads in the lower thigh region 103 and buttock region 104 are higher than heights of the pads in the mid-thigh region. In other words, the pads in the upper thigh region 101 have the lowest heights, the pads in the mid-thigh region 102 have intermediate heights, and the pads in the lower thigh region 103 and the buttock region 104 have the highest heights. The pads in the lower thigh region 103 may have the same heights as the pads in the buttock region 104, or alternatively they may have different heights.

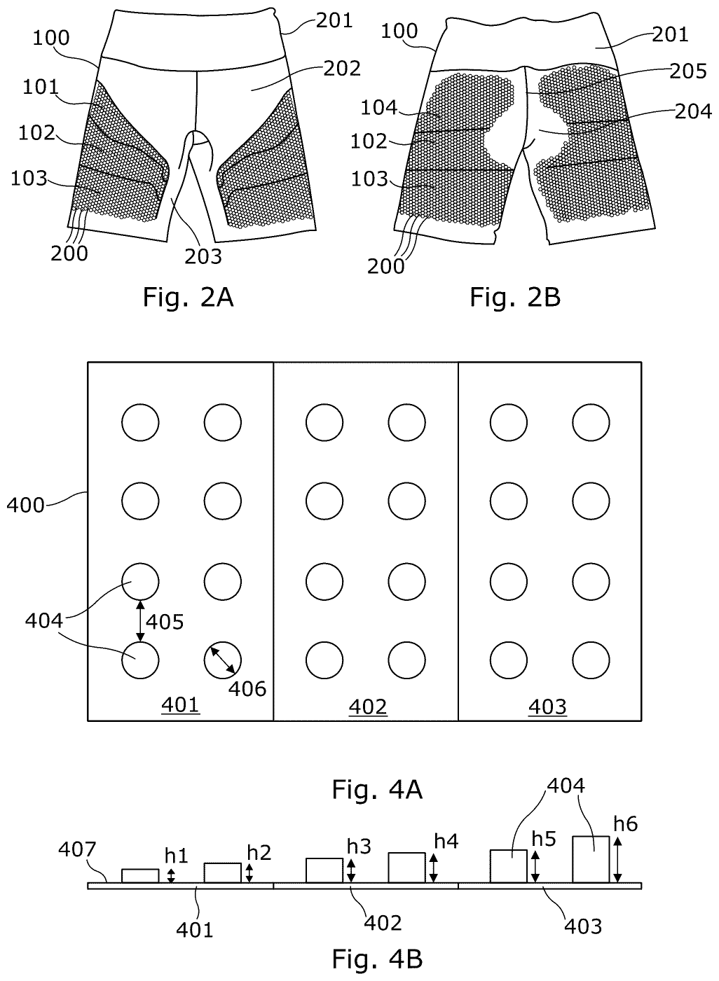

FIGS. 2A and B show front and back views respectively of a compression garment 100 according to the present invention. The garment 100 is illustrated inside-out to show the inner surface 201 and the regions with respective pluralities of pads thereon. The upper thigh region 101, mid-thigh region 102, lower thigh region 103 and buttock region 104 each comprise a plurality of pads 200 as described above. In the illustrated embodiment, each pad is preferably (substantially) cylindrical and preferably has a diameter of 2-10 mm. Pads may be present in any region 101-104 of differing sizes and shapes, or heights. The plurality of pads 200 in the upper thigh region 101 preferably have heights between 0.3-0.5 mm, measured from the base of a pad to the highest point on that pad. Similarly, the plurality of pads in the mid-thigh region 102 preferably have heights between 0.5-1 mm and the pluralities of pads in the lower thigh region 103 and buttock region 104 preferably have heights between 0.9-1.5 mm. The pads 200 may deform when the garment is stretched, or when they are pressed into the wearer's skin. It is preferable the pads are somewhat soft and/or resilient, rather than being fully rigid and hard because hard/rigid pads may make the garment more difficult to stretch, reducing wearer comfort and leading to undesirable local compression pressures.

The pads 200 may be screen-printed ink-polymer-based (for example, using a graduated high build screen printing process), but other methods of creating slightly raised pads may also be used. When using a screen-printing process, the height of the pads can be adjusted by altering the number of ink layers applied to the fabric. For example, the lowest pads (in the upper thigh region) may be printed using one layer of ink, the intermediate pads (in the mid-thigh region) may be printed using two layers of ink, and the highest pads (in the upper thigh region) may be printed using three layers of ink. The pads may be placed directly on to finished garments 100, or for example, be pre-placed/printed/fixed on to flat fabric that is subsequently cut into desired shapes to be attached in turn to the finished garment 100 by means for example of, sewing, gluing, or ultrasonic welding.

In some circumstances, if the pads 200 are spaced too closely, the garment 100 may become less comfortable to wear, as sweat is unable to escape as quickly 1o through the spaces between pads 200. Accordingly in an example, the separation distance between the edges of closest neighbouring pads 200 may be between 1-5 mm.

In some circumstances, if the pads 200 are spaced too far apart, the garment 100 may become less effective at managing cellulite. Accordingly, in an example the separation distance between the edges of closest neighbouring pads 200 may be between 1-5 mm. In other examples, the separation distance is between 1.5-3 mm. As will be discussed below, the spacing between neighbouring pads 200 may be varied above the minimum separation distance so as to, for example, provide reduced pad density in some regions.

The pads 200 preferably do not follow the surface mapping of named, superficial veins (alongside which, run the superficial lymphatic vessels). Unpadded regions (smooth regions that do not have any pads 200) will preferably be left in regions near any named, superficial veins (so avoiding any named superficial lymphatic vessel(s)) to encourage lymphatic drainage from the smallest lymphatic vessels and plexuses towards the larger ‘collecting’ lymphatics and so on towards the regional lymph nodes.

The inner surface 201 of the garment 100 is left without pads in a groin region 202, an inside thigh region 203, a crotch region 204, and natal cleft region 205 (which cover the wearer's groin, inner thigh, crotch and natal cleft respectively). These unpadded regions 202-205 are smooth and do not comprise pads 200. The groin region 202 is left without pads so that the inguinal (regional) nodes in the groin are not compressed, which could inhibit the drainage of lymph from the thighs and buttock into the inguinal nodes. Similarly, the inside thigh region 203 does not comprise pads so as to avoid compressing the greater saphenous vein, which could inhibit blood flow through the wearer's legs. The crotch region 204 and natal cleft region 205 are left without pads 200 to increase wearer comfort. However, it should be noted that the garment 100 would still function (but less effectively) if one of these more regions did comprise pads.

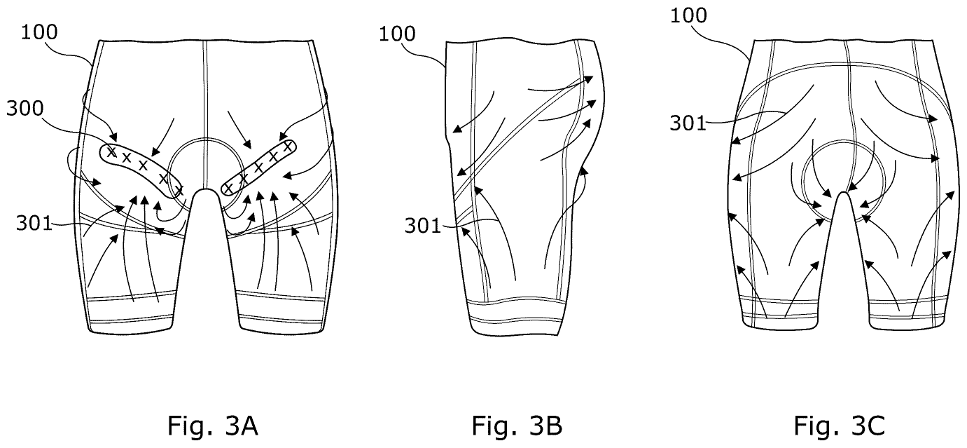

FIGS. 3A, B, and C show front, side, and rear views respectively of the compression garment 100 of FIG. 1, with arrows 301 illustrating the overall pattern of drainage paths of lymph in the wearer's body towards the inguinal (regional) nodes 300 in the groin. Lymph in a human's thigh, groin and buttock regions ideally drains towards the inguinal nodes 300 following the drainage paths illustrated by the arrows 301. The compression garment 100 acts to direct and encourage lymph egress along the natural drainage paths in the wearer's body, thereby preventing the effects of poorly draining lymph in the thigh, groin and buttock, and helping prevent or reduce cellulite formation. This is achieved by having pluralities of pads with greater heights in regions located further from the inguinal nodes 300 along the lymph drainage paths indicated by the arrows 301, and pluralities of pads with lower heights in regions located closer to the inguinal nodes 300 along the lymph drainage paths.

With reference to FIGS. 2A-B in combination with FIGS. 3A-C, when the compression garment 100 is worn, the pads 200 act to increase the flow of lymph along the arrows 301 indicating the lymph drainage paths and into the inguinal nodes 300. The plurality of pads 200 in each region presses against the wearer's skin in that region. Higher pads create a higher local pressure in the wearer's skin and dermis. The heights of the plurality of pads in each region are proportional to the length of the lymph drain path for that region, as indicated by the distance to the inguinal nodes 300 along the arrows 301 indicating the lymph drainage paths. For example, the lower thigh region 103 is located further from the inguinal nodes 300 in the groin, than the upper thigh region 102 is. As indicated by the arrows 301, this means the lower thigh region 103 has a longer lymph drainage path than the upper thigh region 101, and consequently the heights of pads 200 in the lower thigh region 103 are greater than the heights of pads 200 in the upper thigh region 101. By encouraging or enhancing the drainage of lymph from the lower thigh and buttock to the inguinal nodes 300, the garment 100 minimises any lymph retention in the groin, thigh and buttock, thereby inhibiting cellulite formation in the first instance, or reducing the progression of cellulite development, or helping minimise the appearance of cellulite already present.

The different heights of pads creates a directional push which encourages and enhances the drainage of lymph, as discussed above. However, it is also envisioned that all the pads on the garment could be of the same height or range of heights. Cellulite mitigation still occurs when all pads have same height, because the pads also enhance the passage of blood through micro-vasculature, encouraging the delivery of well oxygenated and nutrient-rich blood.

The cellulite-reduction effect does not disappear when the garment 100 is removed. Instead, cellulite formation is reduced for a period of time, which will generally be proportional to the length of time that the garment 100 is worn. Therefore, after wearing the garment for a first period of time, the wearer can then remove the garment and display cellulite-minimised/free skin for a second period of time. The cellulite-reduction effect may persist for a similar time period to the length of time that the garment 100 was previously worn. For example, wearing the garment 100 overnight as or underneath nightwear may result in cellulite reduction for a good part of the following day, and wearing the garment during the morning may result in a reduction in the appearance of cellulite over the following afternoon. The cellulite reduction effect caused by wearing the garment may also be cumulative. For example, wearing the garment 100 overnight for a week may result in cellulite reduction during the following week, even if the garment 100 is not worn during that following week.

The garment 100 is suitable for wearing as underwear underneath other clothes, so that the pluralities of pads press directly against the wearer's skin. Alternatively, the garment 100 may be worn with underwear underneath, for reasons of comfort or hygiene. The heights and rigidity of the pads are sufficient that they press into and compress the skin even through the material of an intermediate garment, so long as the interspacing garment is not excessively thick. Preferably, if underwear is to be worn under the garment 100, it should be a type that minimally overlaps the padded regions 101-104—for example, a thong.

FIGS. 4A and 4B show a top view and side view respectively of a simplified model of a garment 400 comprising a plurality of regions, each region comprising a plurality of pads 404 on the inner surface 407. Three regions 401, 402, and 403 on the garment 400 each comprise a plurality of pads 404. The plurality of pads 404 in each region 401, 402, 403 are distributed in an ordered pattern over the region, as shown in FIGS. 4A and 4B. Alternatively, the plurality of pads 404 in a region could be distributed stochastically, meaning that the pads 404 are disturbed randomly over the region. The closest separation distance 405 between the edges of neighbouring pads 404 may vary, for example due to manufacturing tolerances, but are preferably between 1-5 mm, and more preferably between 1.5-3 mm.

The pads 404 are each a cylindrical shape with diameter 406. While the illustrated pads 404 each has approximately the same diameter, the pads 404 could alternatively have different diameters. Heights of the plurality of pads 404 vary between the regions 401, 402 and 403. In region 401, the plurality of pads 404 have heights h1 and h2. In region 402, the plurality of pads 404 have heights h3 and h4, and in region 403, the plurality of pads 404 have heights h5 and h6. Heights h1 and h2 are less than heights h3-h6. Heights h3 and h4 are greater than heights h1 and h2 and less than heights h5 and h6. Heights h5 and h6 are greater than heights h1-h4. In alternative embodiments, the heights of the plurality of pads in each region could be identical (within manufacturing tolerances). With reference to the compression garment 100 in FIGS. 1A-C and 2A-B, region 401 on the inner surface 407 would correspond to the upper thigh region 101 on the inner surface 201, region 402 to the mid-thigh region 102, and region 403 to the lower thigh region 103 and/or the buttock region 104 (although it should be understood that each of these regions would have far more pads in practice and would have a different shape/layout).

While the illustrated pads 404 are (substantially) cylindrical, the pads could alternatively have different shapes. For example, they could be domed-shaped, U-shaped, tapered, straight-sided, or any other shape that forms a protrusion from the inner surface of a garment. Cylindrically shaped pads are advantageously easier to manufacture. Alternatively, pads could have for example, an oval-shaped or hexagonal footprint. The garment could comprise differently shaped pads in different regions of the garment, or a mixture of pad shapes within a particular region.

The garment 100 may comprise an elastic material such as those commonly known in the art for use in cycling shorts, for example fabrics created through use of elastane/Lycra® woven in to a ‘base fabric’ that is typically made of a synthetic yarn such as polyester, or polyamide (natural yarns may be used in the base fabric also, such as cotton, bamboo, or silk for example). This ensures that the garment 100 follows the contours of the wearer's body. Additionally, the elastic material causes the garment 100 to exert a compressive pressure (force) on the user's skin, in addition to the local pressures generated by the pads 200. Advantageously, the compression may also improve the wearer's blood circulation. Preferably, the garment 100 is made using compression fabrics with high breathability, further enhancing wearer comfort over long periods. For example, cotton-rich compression fabrics would be suitable.

A compression level of the garment 100 may be between 1 mmHg and 50 mmHg, more preferably between 3 mmHg and 15 mmHg (similar to standard cycling shorts and other compression athletic gear).

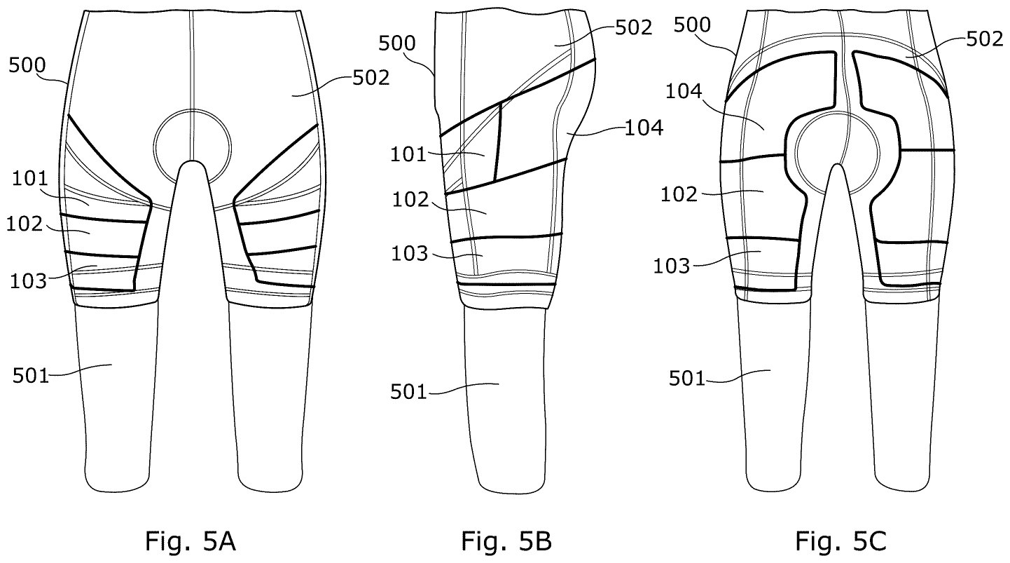

FIGS. 5A-C show an alternative embodiment of a compression garment 500 in the form of a pair of leggings or trousers, comprising at least one lower-leg portion 501 extending beyond an upper portion 502 (which includes the regions 101-104 comprising pluralities of pads). The lower-leg portion 501 may extend all the way to the wearer's feet/ankles so as to form a set of long trousers, tights, leggings or jeggings. Alternatively, the lower leg portion 502 may extend beyond the upper portion 502 over the knees, or some distance down the calf, so as to form a set of longer shorts/capris. Further alternatively, the upper portion 502 could extend upwards so as to cover part of the wearer's midriff and/or act as a waistband. The lower-leg portion 501 may be made of the same material as the upper portion 502, or alternatively it may be made of a different material. For example, the lower-leg portions 502 extending below the knee could be made of a looser, less elasticated material, as compression of the wearer's skin is not required in this region. The lower-leg portions 502, may also not comprise padded regions as these areas are not usually affected by cellulite. Such a garment 700 could be more comfortable for wearing over an extended period of time, for example as pajamas or leggings. The compression garment 500 could be another type of apparel, for example: cropped leggings, ‘jeggings’, ‘cycle shorts’, briefs/pants, or tights.

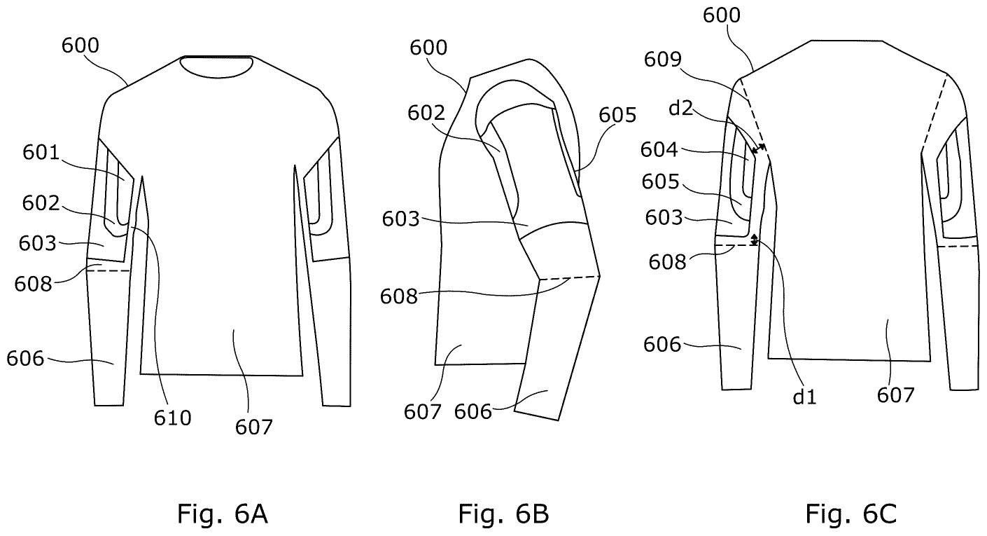

FIGS. 6A, B and CB show a front, side, and back view respectively of a compression garment 600 configured to be worn over at least a wearer's shoulders and upper arms. The garment 600 in this embodiment is a long-sleeve t-shirt, which covers the wearer's body from the shoulders down to the top of the wrists and the bottom of the torso.

Five padded regions are illustrated on the inner surface of the garment 600. An upper inner biceps region 601 covers the wearer's upper inner biceps, i.e. the inside front of the wearer's upper arm. Similarly, an upper inner triceps region 604 covers the wearer's upper inner triceps, i.e. the inside rear of the wearer's upper arm. An outer arm region 603 covers the side of the upper arm, wrapping around from the front to the back of the arm so as to cover the lower biceps on the front of the arm and the lower triceps on the rear of the arm. A mid-biceps region 602 is located between the upper inner biceps region 601 and the outer arm region 603, and a mid-triceps region 605 is similarly located between the upper inner triceps region 604 and the outer arm region 603. As with the compression garment 100 of FIGS. 1A-C, alternative embodiments are envisioned in which one or more of the regions 601-605 are larger or smaller and/or have different shapes (for example the upper inner biceps region 601 may be extended to cover the whole of the wearer's inner biceps, rather than just the upper inner biceps). In addition, one or more the regions (in particular the mid-biceps region 602 or mid-triceps region 605) could be omitted in alternative embodiments, and the size and shape of the remaining regions could be adjusted accordingly (e.g. the upper inner biceps region 601 may be extended to the outer arm region 603). As with the compression garments 100 for the wearer's legs discussed above, the illustrated regions are symmetric on the wearer's left and right sides.

Each padded region 601-605 comprises a plurality of pads (the pads themselves are not shown in FIGS. 6A-C) on the inner surface of the garment 600. The regions 601-605 may be contiguous as shown in FIGS. 6A-C, or alternatively some of the regions 601-605 may be separated from each other by spaces which do not have pads. The padded regions 601-605 stop before the line of the elbow joint 608, with the closest distance d1 from the lower edge of the padded regions 601-605 to the line of the elbow joint 608 being at least 3 cm. This ensures that the garment 600 remains comfortable when the wearer's elbows are bent. Similarly, the padded regions stop before the line of the arm pit 609, with the closest distance d2 from the upper edge of the padded regions 601-605 to the line of the elbow joint 608 being at least 5 cm. This ensures the garment 600 remains comfortable through the range of motion of the wearer's shoulders.

Heights of the pads in the upper inner biceps region 601 are lower than heights of the pads in the mid-biceps region 602, and heights of the pads in the upper inner triceps region 604 are lower than heights of the pads in the mid-triceps region 605. Heights of the pads in the outer arm region 603 are higher than heights of the pads in the mid-biceps region 603 and mid-triceps region 605. In other words, the pads in the upper inner biceps region 601 and upper inner triceps region 604 have the lowest heights, the pads in the mid-biceps region 602 and mid-triceps region 605 have intermediate heights, and the pads in the outer arm region 603 have the highest heights. The pads in the upper inner biceps region 601 may have the same heights as the pads in the upper inner triceps region 604, or alternatively they may have different heights. Similarly, the pads in the mid-biceps region 602 may have the same or different heights to pads in the mid-triceps region 605.

The shapes, positioning and heights of pads in the lowest-height, highest-height and intermediate height regions in the garment 600 may be the same as described for the lower-leg compression garment 100. Alternatively, the pads may have a different set of heights.

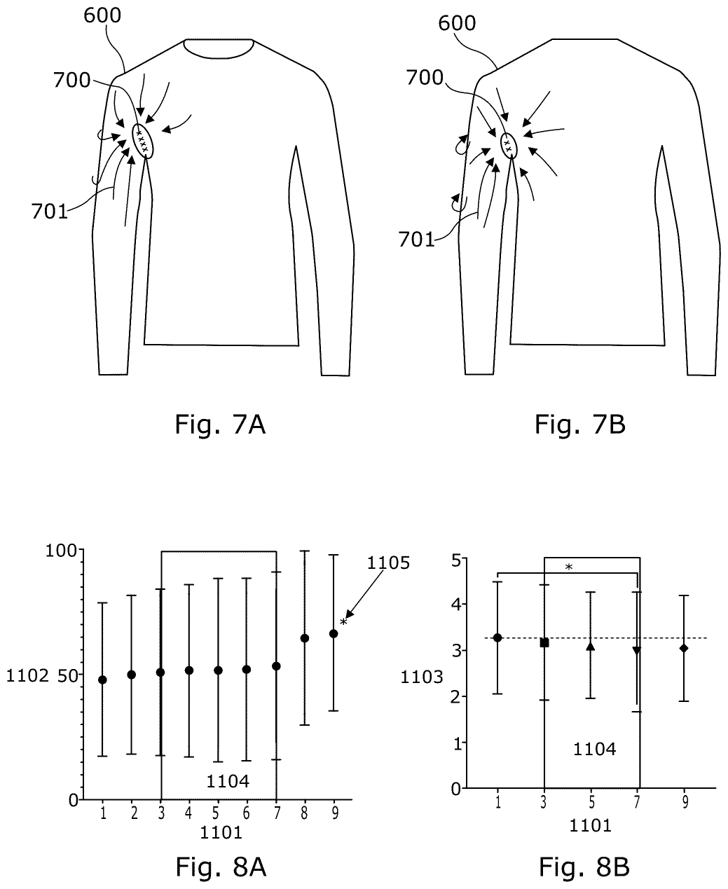

In the embodiment illustrated in FIG. 6A-C, the compression garment 600 is a long sleeve top, with lower arm regions 606 extending below the line of the elbow joint 608. Alternatively, the compression garment 600 could be a short-sleeve top, i.e. without regions extending below the line of the elbow joint 608. As with the lower-leg portions 502 of the compression garment 500 shown in FIGS. 5A-C, the lower-arm portion 606, and torso portion 607 are unpadded regions, which do not comprise pads, as these areas are not usually affected by cellulite. The inside arm region 610 is also unpadded, with a width (i.e. the closest separation distance between the inside edges of the padded regions on the front of the arm and the inside edges of the padded regions on the rear of the arm) of at least 6 cm. This ensures that the key axillary blood vessels in the wearer's inside upper arm are not compressed, which could impede the wearer's blood (and lymph) flow. FIGS. 7A and B show front and back view respectively of the compression garment 600 of FIG. 6, with arrows 701 illustrating the overall pattern of drainage paths of lymph in the wearer's body towards the axillary (regional) lymph nodes 700 in the armpit. Lymph in a human's upper arm and shoulder drains towards the axillary nodes 700 following the drainage paths illustrated by the arrows 701. The compression garment 600 acts to direct and encourage lymph egress along the natural drainage paths in the wearer's body, thereby preventing the effects of poorly draining lymph in the upper arm, and shoulder, and helping prevent or reduce cellulite formation and progression. As with the compression garment for the lower body, which has been described previously, this is achieved by having pluralities of pads with greater heights in regions located further from the axillary nodes 700 along the lymph drainage paths indicated by the arrows 701, and pluralities of pads with lower heights in regions located closer to the axillary nodes 700 along the lymph drainage paths.

With reference to FIGS. 6A-C, when the compression garment 600 is worn, the pads act to increase the flow of lymph along the arrows 701 indicating the lymph drainage paths and into the axillary nodes 700. By encouraging or enhancing the drainage of lymph from the bottom of the upper arm to the axillary nodes 700 (ie. from more distal to more proximal), the garment 600 inhibits lymph retention in the upper arm and shoulder, thereby inhibiting cellulite formation in the first instance, or reducing the progression of cellulite development, or helping minimise the appearance of cellulite already present.

As with the compression garment configured to be worn over at least a wearer's pelvic area and upper legs discussed above, it is also envisioned that all the pads on the garment could be of the same height or range of heights. Cellulite mitigation still occurs when all pads have the same height, because the pads also enhance the passage of blood through the micro-vasculature, encouraging the delivery of well oxygenated and nutrient-rich blood.

The illustrated compression garment 600 is in the form of a long-sleeve top. Alternatively, the compression garment 600 could be another type of apparel, for example: t-shirts, polo shirts, cycling vest, jumpers, sweaters, fleeces, shirts, or blouses.

### Example

A study was performed in which skin/dermis oxygenation levels of patients' legs were measured for garments with and without ink-polymer pads. The patients each wore a version of the padded compression garment 100 shown in FIG. 2.

28 participants were placed in the supine position (supine 1) for five minutes. They were then moved to an upright seated position for five minutes. Then they returned again to supine (supine 2) for another five minutes (15 minutes total recorded).

Skin probes were placed to measure:
Blood flux
SO2
Oxyhaemoglobin (Oxy-Hb)
Deoxyhaemoglobin (De-oxyHb)

The Moor Instruments Ltd VMS software was used to obtain the measurements. Ranges of Interest (ROI) provided the mean for each measurement. The process was undertaken on limbs without a padded garment and next, repeated with the padded garment. A five-minute recording (baseline 2) was also taken after the garment had been removed for 10 min.

The results are collated in Table 1 below. As shown, the padded compression garment showed statistically significantly different values when compared to controls (non-padded garment) for: SO2 when sitting; Oxy-Hb when sitting; De-oxyHb when supine, sitting and supine again, slope and time to reach max. These results demonstrate that when worn, the padded garment stimulates the enhanced delivery of Oxy-Hb to the skin and dermis beneath and aids the removal of De-oxyHb from those same tissues.

As previously discussed, considered factors contributing to cellulite formation include: (1) micro-vascular (capillary) dysfunction can lead to a poorer supply of Oxy-Hb to the skin and subcutaneous tissues (promoting fibrin deposition, fibrosis and fluid retention), and (2) poor lymph (comprising protein-rich fluid and cellular ‘waste products’) drainage can contribute to the development of generalised tissue fibrosis and in particular, to those fibrous bands that form between subcutaneous fat (lipid-rich) cells (lipocytes). By enhancing the delivery of Oxy-Hb and removal of De-oxyHb from the skin, padded garments according to the present invention are shown to improve micro-vascular function and thereby help to both improve the appearance of already established cellulite and slow or prevent cellulite progression.

TABLE 1

| | | | | | | | | 95% | | |
|---|---|---|---|---|---|---|---|---|---|---|
| | | | Control | | Padded | | | Confidence | | |
| | | | Mean | SD | Mean | SD | Delta | 1 | 2 | p |
| Flux | Left | Supine 1 | 14.82 | 2.07 | 14.29 | 1.65 | 0.53 | −2.36 | 3.43 | 0.709 |
| | | Seated | 7.60 | 0.65 | 8.54 | 0.97 | −0.94 | −2.40 | 0.51 | 0.194 |
| | | Supine 2 | 12.45 | 0.88 | 13.47 | 1.42 | −1.01 | −3.27 | 1.23 | 0.363 |
| | Right | Supine 1 | 12.20 | 1.31 | 13.40 | 1.97 | −1.19 | −3.23 | 0.84 | 0.240 |
| | | Seated | 7.32 | 0.65 | 8.29 | 1.26 | −0.97 | −2.09 | 0.49 | 0.086 |
| | | Supine 2 | 11.82 | 1.25 | 12.96 | 1.92 | −1.43 | −3.11 | 0.83 | 0.245 |
| S02 | Left | Supine 1 | 34.10 | 2.40 | 34.64 | 2.62 | −0.54 | −3.51 | 2.43 | 0.712 |
| | | Seated | 18.75 | 2.09 | 24.10 | 2.33 | −5.35 | −8.23 | −2.47 | <0.001 |
| | | Supine 2 | 36.06 | 2.15 | 34.89 | 2.60 | 1.22 | −1.82 | 4.27 | 0.419 |
| | Right | Supine 1 | 36.23 | 2.57 | 34.89 | 2.80 | −0.66 | −3.97 | 2.65 | 0.686 |
| | | Seated | 19.89 | 2.09 | 24.19 | 2.22 | −4.30 | −7.45 | −1.15 | <0.001 |
| | | Supine 2 | 37.51 | 2.70 | 37.58 | 2.85 | −0.08 | −3.52 | 3.37 | 0.965 |
| OxyHb | Left | Supine 1 | 9.68 | 1.55 | 10.06 | 2.07 | −0.38 | −2.51 | 1.75 | 0.719 |
| | | Seated | 7.91 | 1.19 | 9.97 | 1.99 | −2.06 | −4.37 | 0.26 | 0.079 |
| | | Supine 2 | 10.68 | 1.45 | 11.05 | 2.23 | −0.37 | −2.57 | 1.83 | 0.419 |
| | Right | Supine 1 | 7.10 | 0.94 | 6.69 | 1.00 | 0.40 | −0.67 | 1.48 | 0.447 |
| | | Seated | 5.69 | 0.70 | 6.22 | 1.11 | −0.54 | −1.69 | 0.62 | 0.348 |
| | | Supine 2 | 7.84 | 0.97 | 7.09 | 1.16 | −0.73 | −0.30 | 1.79 | 0.157 |
| DeoxyHb | Left | Supine 1 | 11.89 | 0.96 | 10.49 | 0.77 | 1.38 | 0.01 | 2.75 | 0.050 |
| | | Seated | 24.50 | 1.74 | 18.38 | 1.51 | 6.13 | 3.86 | 8.39 | <0.001 |
| | | Supine 2 | 17.02 | 1.48 | 17.12 | 1.87 | −0.11 | −2.52 | 2.31 | 0.930 |
| | | Slope | 0.16 | 0.02 | 0.15 | 0.02 | 0.02 | −0.01 | 0.04 | 0.149 |
| | | Time to | 254.43 | 8.34 | 244.64 | 7.53 | 9.78 | −8.54 | 28.12 | 0.283 |
| | | Max | | | | | | | | |
| | Right | Supine 1 | 11.86 | 0.96 | 10.49 | 0.77 | 1.38 | 0.01 | 2.75 | 0.050 |
| | | Seated | 24.50 | 2.89 | 18.05 | 2.70 | 6.13 | 3.86 | 8.39 | <0.001 |
| | | Supine 2 | 12.52 | 0.92 | 10.68 | 0.86 | 1.84 | 0.43 | 3.25 | 0.001 |
| | | Slope | 0.14 | 0.02 | 0.11 | 0.02 | 0.03 | 0.01 | 0.05 | 0.002 |
| | | Time to | 256.75 | 9.05 | 224.39 | 10.89 | 32.35 | 13.90 | 50.81 | 0.001 |
| | | Max | | | | | | | | |

### Second Example

A second study was performed to assess the long-term effects of using compression garments comprising variable height-pads. The apparel used was a version of the compression garment 100 shown in FIG. 2.

18 females were selected as participants for the study, with the participants having a mean age of 47.5 years and an age range of 26-60 years. Prior to the study, all of the participants were affected by cellulite to varying degrees. Their level of cellulite was assessed and graded as the first step of the study. Three participants had Grade 1 cellulite, meaning that their skin was smooth whilst the participant is standing/lying, but alterations can be seen by pinching the skin or by muscle contraction. Seven participants had Grade 2 cellulite, meaning that an ‘orange-peel’ or mattress appearance is evident on their skin upon standing without the use of any manipulation (pinching or muscle contraction). Eight participants had Grade 3 cellulite, meaning that the alterations seen in Grade 2 presented together with raised areas and nodules.

The trial was arranged in 3 stages, lasting 9 weeks overall, designed to assess the before, during and after-effects of the low-compression apparel with its integrated, variable-height pads. Weeks 1 and 2 were a stabilisation period, during which participants wore regular (i.e. unpadded, non-compression) undergarments. During the active period of weeks 3 to 7, the participants wore the compression garment. Finally, during weeks 8 and 9, the participants resumed wearing their regular undergarments.

The participants were assessed by clinicians during each week of the trial. During each appointment, several measurements and assessments were made: standard demographics, weight, height, and any changes to dietary or exercise regimens over the trial period; subjective assessment of the cellulite by the participant; objective assessment and grading using the cellulite grading system described above; taking images of their cellulite-affected areas (this may include an area over the buttocks and an area over the thighs); and using a 3D high-resolution medical imaging system (the Vectra H2 imaging system) to assess the volumetric water content of tissue in of the participant's cellulite-affected areas. The images and results of the imaging system were independently assessed and graded by a panel of clinicians for objective classifications.

The results of the trial are shown in FIG. 8. The x-axis of FIGS. 8A and 8B is the week 1101 of the study. During the active period 1104 (highlighted on the graphs) between weeks 3 and 7, the participants wore the compression garment.

FIG. 8A shows the participant's subjective assessment 1102 of their own cellulite on the y-axis, with 100% indicating maximum satisfaction (i.e. minimisation of discolouration/alteration due to cellulite), 0% indicating minimum satisfaction, and 50% indicating no change. Taking the 3D measurements from the imaging system, FIG. 8B shows the volume of water 1103 in cubic cm on the y-axis. Higher water content indicates the presence of more lymph in the tissue. An average was calculated for each week from all participant's measurements, with error bars also shown on the graphs. The data points marked with asterisks 1105 are statistically significant.

FIGS. 8A and 8B indicate a statistically significant improvement in both subjective and objective metrics caused by wearing the compression garment. Participant assessment of their cellulite improved by 18.5% in just 4 weeks, indicating significant user satisfaction. Images from the study showed that cellulite dimples in the participant's thighs reduced significantly in volume over the 4 weeks the compression garments were worn. As shown by FIG. 8B, there was a significant decrease in water (and therefore lymph) under the skin in the participant's tissue over the course of wearing the compression garments. The subjective improvement and volumetric water reduction in the tissue persisted in weeks 7 and 9 once the participants stopped wearing the compression garments, demonstrating the persistent long-term benefits of the compression garments on cellulite reduction.

As previously discussed, using pads of differential height in different regions of a garment can enhance the drainage of lymph from the lower thigh and buttock to the lymph nodes. A similar end-effect can be achieved by using different inter-pad spacings in different regions of a garment (were pads to be of an equal height). Higher pad heights can create higher local pressure enhancements which encourage lymph to flow into regions of lower local pressure distortion (that is, regions with no or lower pad heights). Local pressure enhancement is also correlated with inter-pad spacing, with wider pad spacing causing higher local pressure enhancement. As a result, lymph can be directed to flow from a region of widely spaced pads into a region of closely spaced pads. An additive end-effect might be expected were pads to be of differential height and with varying inter-pad spacing. This effect would be expected however, to be limited were the inter-pad spacing to be too wide, for example to be significantly greater than 6 mm (when the maximum pad height is for example at 1.5 mm).

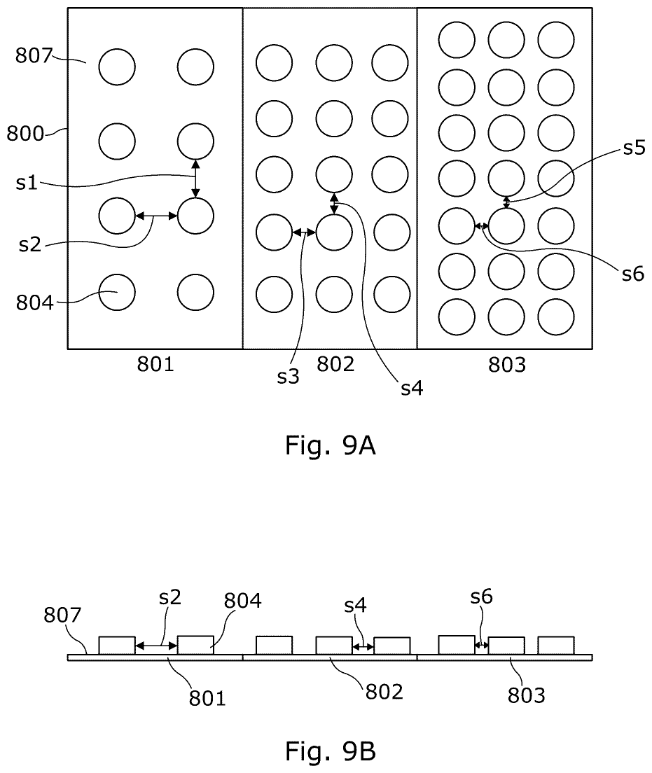

FIGS. 9A and 9B show a top view and side view respectively of a simplified model of a garment 800 comprising a plurality of regions, each region comprising a plurality of pads 804 on the inner surface 807. Three regions 801, 802, and 803 on the garment 800 each comprise a plurality of pads 804. The plurality of pads 804 in each region 801, 802, 803 are distributed in an ordered pattern over the region. In other examples, the plurality of pads 804 in a region could be distributed stochastically, meaning that the pads 804 are disturbed randomly over the region.

The pads 804 are each a cylindrical shape with diameter 806, but as previously discussed in relation to FIG. 4, in some examples pads 804 could alternatively have different diameters. Inter-pad spacing varies between each of the regions 401, 402, 403. The inter-pad spacing is defined herein as the closest separation distance between the edges of neighbouring pads 804. In the region 801, the pads have a minimum inter-pad spacing s1 and maximum inter-pad spacing s2. In the region 801, the pads have a minimum inter-pad spacing s3 and maximum inter-pad spacing s4. In the region 801, the pads have a minimum inter-pad spacing s5 and maximum inter-pad spacing s6. Spacings s1 and s2 are closer than spacings s3-s6. Spacings s3 and s4 are wider than spacings s1 and s2 and closer than spacings s5 and s6. Spacings s5 and s6 are wider than spacings s1-s4. In alternative embodiments, the inter-pad spacings of the plurality of pads in each region could be identical (within manufacturing tolerances).

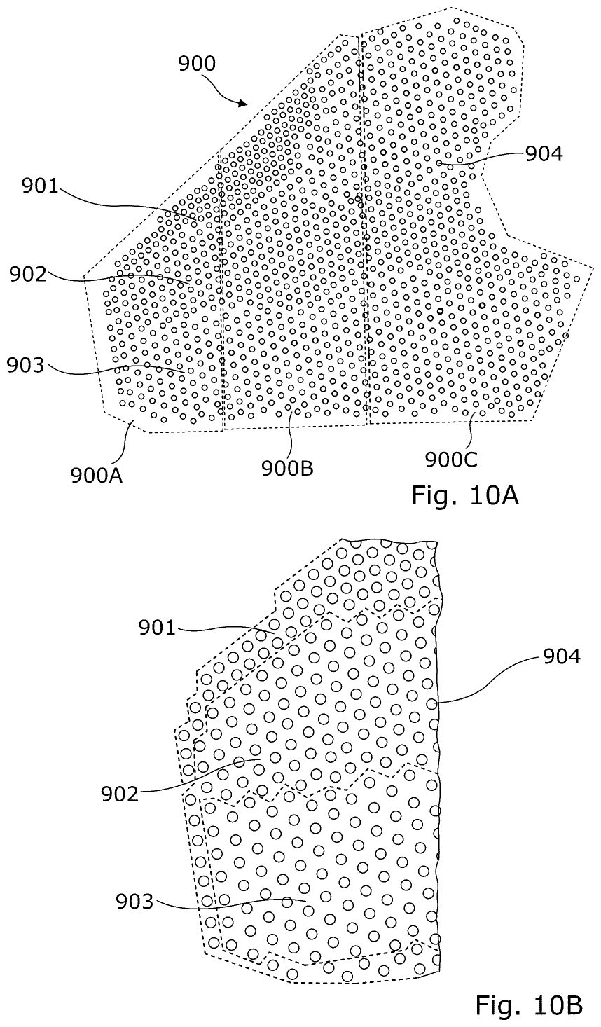

FIGS. 10A and 10B show a template 900 for screen-printing onto the inner surface of a compression garment, such as the compression garment 100 of FIG. 1. The template 900 is shown flat to allow the location of pads 804 to be more easily discerned. The template 900 corresponds to the right leg of the garment. In some examples, a mirror-image version of the template 900 is used for the left leg of the garment. It can be seen that when printed onto the inner surface of the compression garment 100, the shape of the template 900 matches the combined shape of the pads regions 101-104 on the compression garment 100. The front portion 900A of the template 900 covers the front of the leg in use, the side portion 900B covers the side of the leg, and the rear portion covers the back of the leg and buttock. That is, the portions 900A-C approximately correspond to the views 1A-C of the compression garment 100 (noting that FIGS. 1A-C shows the regions from the outside of the garment, whereas FIG. 900 shows the inner side.)

The inter-pad spacings on the upper thigh region 901 are closer than the inter-pad spacings in the lower thigh region 903 and buttock region 904. The inter-pad spacings 902 mid-thigh region are wider than the inter-pad spacings in the upper thigh region 901, but closer than the inter-pad spacings in the lower-thigh region 903 and buttock region 904. That is, with reference to FIGS. 9A and 9B, region 801 on the inner surface 807 would correspond to the upper thigh region 901, region 802 to the mid-thigh region 902, and region 803 to the lower thigh region 903 and/or the buttock region 904. In the example shown, the average inter-pad spacing between pads in the upper thigh region 901 is 3 mm, the average inter-pad spacing between pads in the mid-thigh region 902 is 4 mm, and the average inter-pad spacing between pads in each of the lower thigh region 903 and buttock 3o region 904 is 5 mm. In other examples, the inter-pad spacings between pads in the upper thigh region 901 are between 2.5 and 3.5 mm, the inter-pad spacings between pads in the mid-thigh region 902 are between 3.5 and 4.5 mm, and the inter-pad spacings between pads in each of the lower thigh region 903 and buttock region 904 are between 4.5 and 5.5 mm.

In some examples, the pads may be located on the compression garment 100 according to the template 900, but with pad heights in the regions 901-904 according to the regions 101-104 in previously described examples. In other words, both the inter-pad spacings and the heights of the pads vary between the regions 901-904, with higher pad heights being spaced with wider inter-pad spacings. In one example, the pads in the upper 1o thigh region 901 have an heights of between 0.3 and 0.5 mm, the pads in the mid-thigh region 902 have heights between 0.5-1.0 mm, and the pads in the lower thigh region 903 and buttock region 904 have an heights between 0.9-1.5 mm.

The pad heights and spacings work synergistically to further enhance directional pushing of lymph. When the compression garment 100 is worn, the material of the garment is stretched and curved around the surfaces of the wearer's limbs and torso. This causes a compression force to be applied radially inwards at each pad. When the pads are more widely spaced around the circumference of the limb or torso, the angle between neighbouring pads is increased. This causes a larger component of the tensile force generated by stretching the garment 100 to be directed radially inwards, enhancing local compression. Conversely, reducing the inter-pad spacing decreases the angle between neighbouring pads, causing a smaller component of the tensile force to be directed radially inwards and reducing local compression

The different inter-pad spacings create a directional push which encourages and enhances the drainage of lymph, as discussed above. However, it is also envisioned that all the pads on the garment could be of the same inter-pad spacings or range of inter-pad spacings. Cellulite mitigation still occurs in examples where all regions have same inter-pad spacings, because as previously discussed the pads also enhance the passage of blood through micro-vasculature, encouraging the delivery of well oxygenated and nutrient-rich blood.

It will be understood that variable spacings may also be used for a compression garment configured to be worn over at least a wearer's shoulders and upper arms. The regions comprising higher pads would alternatively or additionally comprise wider inter-pad spacings, and the regions comprising lower pads would alternatively or additionally comprise closer inter-pad spacings.

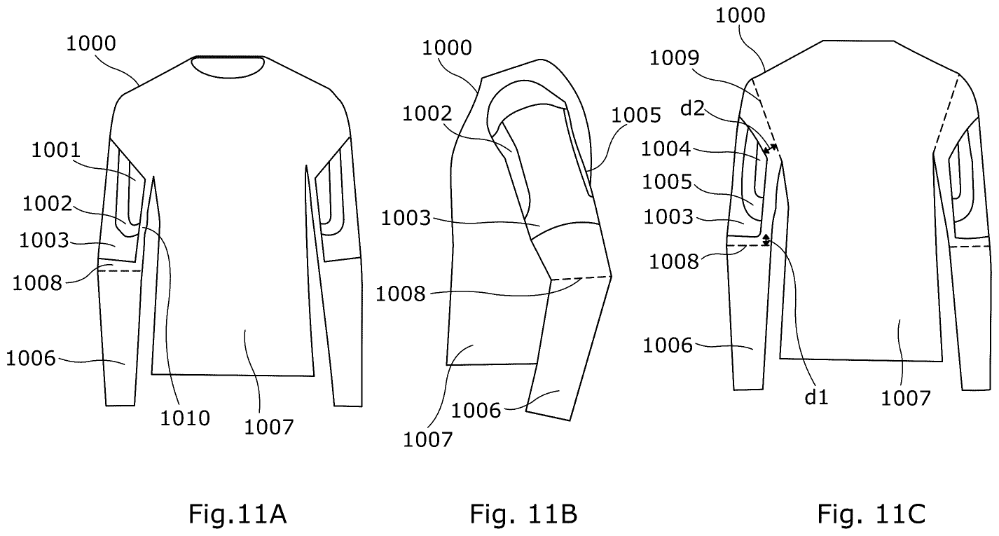

An exemplary compression garment 1000 comprising variable inter-pad spacings can be described in relation to FIG. 11A to 11C, which has the same physical dimensions as the compression garment 600 of FIG. 6. The compression garment 1000 comprises five padded regions 1001-1005, which have the same terminology, shapes, and positions as the five padded regions 601-605 on the compression garment 600 in FIG. 6. That is, an upper inner biceps region 1001 covers the wearer's upper inner biceps, an upper inner triceps region 1004 covers the wearer's upper inner triceps, an outer arm region 1003 covers the side of the upper arm, a mid-biceps region 1002 is located between the upper inner biceps region 1001 and the outer arm region 1003, and a mid-triceps region 1005 is similarly located between the upper inner triceps region 1004 and the outer arm region 1003. As with the compression garment 100 of FIGS. 1A-C, alternative embodiments are envisioned in which one or more of the regions 1001-1005 are larger or smaller and/or have different shapes. As per the regions 601-605 of FIG. 6, the padded regions 1001-1005 may be contiguous or separated from each other, and may be positioned so that they stop before the line of the elbow joint 1008, with the closest distance d1 from the lower edge of the padded regions 1001-1005 to the line of the elbow joint 1008 being at least 3 cm. Similarly, the padded regions 1001-1005 may stop before the line of the arm pit 1009, with the closest distance d2 from the upper edge of the padded regions 1001-1005 to the line of the elbow joint 1008 being at least 5 cm.

Inter-pad spacings of the pads in the upper inner biceps region 1001 are closer than inter-pad spacings of the pads in the mid-biceps region 1002, and inter-pad spacings of the pads in the upper inner triceps region 1004 are closer than inter-pad spacings of the pads in the mid-triceps region 1005. Inter-pad spacings of the pads in the outer arm region 1003 are higher than inter-pad spacings of the pads in the mid-biceps region 1003 and mid-triceps region 1005. In other words, the pads in the upper inner biceps region 1001 and upper inner triceps region 1004 have the closest inter-pad spacings, the pads in the mid-biceps region 1002 and mid-triceps region 1005 have intermediate inter-pad spacings, and the pads in the outer arm region 1003 have the widest inter-pad spacings. The pads in the upper inner biceps region 1001 may have the same inter-pad spacings as the pads in the upper inner triceps region 1004, or alternatively they may have different inter-pad spacings. Similarly, the pads in the mid-biceps region 1002 may have the same or different inter-pad spacings to pads in the mid-triceps region 1005.

When the compression garment 1000 is worn, the pads act to increase the flow of lymph along the lymph drainage paths and into the axillary nodes, as previously described in relation to FIGS. 6 and 7. The shapes, positioning, and inter-pad spacings of pads in the widest, closest, and intermediate inter-pad spacings regions in the garment 1000 may be the same as described for the template 900 for a lower-leg compression garment. Alternatively, the pads may have a different set of inter-pad spacings.

The compression garment 1000 may comprise unpadded regions 1006, 1007, and 1010, corresponding to the unpadded regions 606, 607, and 610 of FIG. 6.

As with the compression garment configured to be worn over at least a wearer's pelvic area and upper legs discussed above, it is also envisioned that all the pads on the garment could be of the same inter-pad spacings or range of inter-pad spacings. Cellulite mitigation still occurs when all pads have the same inter-pad spacings, because the pads also enhance the passage of blood through the micro-vasculature, encouraging the delivery of well oxygenated and nutrient-rich blood. It is also envisioned that both the heights and the inter-pad spacings of the pads on the compression garment 1000 may vary between the padded regions 1001-1005. For example, the regions with widest inter-pad spacings (such as the outer upper arm region 1003) may also comprise higher pads, the regions with the closest inter-pad spacings (such as the upper inner biceps region 1001 and upper inner triceps region 1004) may comprise lower pads, and the regions with intermediate inter-pad spacings (such as the mid-biceps region 1002 and mid-triceps region 1005) may comprise intermediate height pads.

1-25. (canceled)

26. A compression garment comprising more than one compression region, an inner surface of each compression region comprising a respective plurality of pads, wherein the inter-pad spacings between each pad in the respective plurality of pads of neighbouring compression regions differs.

27. The compression garment according to claim 26 wherein the inter-pad spacings are between 2.5 mm and 5.5 mm.

28. The compression garment according to claim 26 wherein the heights of each pad in the respective plurality of pads of neighbouring compression regions differs, preferably wherein the heights of each pad are between 0.2 mm and 1.5 mm.

29. The compression garment according to claim 26 wherein the garment is configured to be worn over at least a wearer's pelvic area and upper legs, wherein the neighbouring compression regions include an upper thigh region, a lower thigh region and a buttock region, preferably wherein the inter-pad spacings of the respective plurality of pads on the upper thigh region are closer than the inter-pad spacings of the respective pluralities of pads in the lower thigh region and the buttock region.

30. The compression garment according to claim 29 wherein the heights of the respective plurality of pads on the upper thigh region are lower than the heights of the respective pluralities of pads in the lower thigh region and the buttock region.

31. The compression garment according to claim 26 wherein the garment is configured to be worn over at least a wearer's shoulders and upper arms, wherein the neighbouring compression regions include an upper inner biceps region, an upper inner triceps region and an outer arm region, preferably wherein the inter-pad spacings of the respective pluralities of pads on the upper inner biceps region and upper inner triceps region are closer than the inter-pad spacings of the respective plurality of pads in the outer arm region.

32. The compression garment according to claim 31 wherein the heights of the respective pluralities of pads on the upper inner biceps region and upper inner triceps region are lower than the heights of the respective plurality of pads in the outer arm region.

33. A compression garment comprising more than one compression region, an inner surface of each region comprising a respective plurality of pads, wherein the heights of each pad in the respective plurality of pads of neighbouring compression regions differs.

34. A compression garment configured to be worn over at least a wearer's pelvic area and upper legs, comprising:
an upper thigh region;
a lower thigh region; and
a buttock region,
wherein an inner surface of each of the upper thigh, lower thigh, and buttock regions comprises a respective plurality of pads, wherein heights of the respective plurality of pads on the upper thigh region are lower than heights of the respective pluralities of pads in the lower thigh region and the buttock region.

35. The compression garment of claim 34, further comprising a mid-thigh region, wherein the mid-thigh region comprises a plurality of pads, and wherein heights of the plurality of pads in the mid-thigh region are higher than the heights of the respective plurality of pads in the upper thigh region and lower than heights of the respective pluralities of pads in the lower thigh region and buttock region, preferably further comprising an unpadded groin region and/or an unpadded inside thigh region and/or an unpadded crotch region and/or an unpadded natal cleft region.

36. A compression garment configured to be worn over at least a wearer's shoulders and upper arms, comprising:
an upper inner biceps region;
an upper inner triceps region; and
an outer arm region,
wherein an inner surface of each of the upper inner biceps, upper inner triceps and outer arm regions comprises a respective plurality of pads, wherein heights of the respective pluralities of pads on the upper inner biceps region and upper inner triceps region are lower than heights of the respective plurality of pads in the outer arm region, preferably further comprising an unpadded torso region, and/or an unpadded inside arm region.

37. The compression garment of claim 36, further comprising a mid-biceps region, wherein the mid-biceps region comprises a plurality of pads, and wherein heights of the plurality of pads in the mid-biceps region are higher than the heights of the respective plurality of pads in the upper inner biceps region and lower than heights of the respective pluralities of pads in the outer arm region, preferably further comprising a mid-triceps region, wherein the mid-triceps region comprises a plurality of pads, and wherein heights of the plurality of pads in the mid-triceps region are higher than the heights of the respective plurality of pads in the upper inner triceps region and lower than heights of the respective pluralities of pads in the outer arm region.

38. A method of manufacturing a compression garment comprising:
providing a compression fabric;
screen-printing respective pluralities of ink polymer pads in regions of the compression fabric to form neighbouring compression regions, wherein the number of layers forming the respective ink polymer pads in each region differs; and
forming the compression fabric into a shape suitable to be worn as a garment.

39. A method of manufacturing a compression garment, comprising:
providing a compression fabric;
screen-printing a first plurality of ink polymer pads in an upper thigh region of the compression fabric, each of the first plurality of ink polymer pads having at least one layer;
screen-printing a second plurality of ink polymer pads in a mid-thigh region of the compression fabric, each of the second plurality of ink polymer pads having at least two layers;
screen-printing a third plurality of ink polymer pads in a buttock region and a lower thigh region of the compression fabric, each of the second plurality of ink polymer pads having at least three layers; and
forming the compression fabric into a shape suitable to be worn over at least a wearer's pelvic area and upper legs.

40. A method of manufacturing a compression garment, comprising:
providing a compression fabric;
screen-printing a first plurality of ink polymer pads in an upper inner biceps region and an upper inner triceps region of the compression fabric, each of the first plurality of ink polymer pads having at least one layer;
screen-printing a second plurality of ink polymer pads in a mid-biceps region and a mid-triceps region of the compression fabric, each of the second plurality of ink polymer pads having at least two layers;
screen-printing a third plurality of ink polymer pads in an outer arm region of the compression fabric, each of the second plurality of ink polymer pads having at least three layers; and
forming the compression fabric into a shape suitable to be worn over at least a wearer's shoulders and upper arms comprising a plurality of padded regions.
